(19) United States
(12) Patent Application Publication
(10) Pub. No.: US 2026/0364276 A1
(43) Pub. Date: Nov. 19, 2026

(54) Inhalation Device

(71) Applicant: Mark Wolf, Brooklyn, NY (US)

(72) Inventor: Mark Wolf, Brooklyn, NY (US)

(21) Appl. No.: 19/259,928

(22) Filed: Jul. 3, 2025

Related U.S. Application Data
(60) Provisional application No. 63/808,042, filed on May 19, 2025.

(51) Int. Cl. A61M 11/00; B05B 17/06

(52) U.S. Cl. CPC A61M 11/005; B05B 17/0676

(57) ABSTRACT

An inhalation device. A housing, a reservoir configured within the housing, an aqueous solution contained within the reservoir, a non-thermal mechanism that aerosolizes the aqueous solution, a power source operably coupled to the non-thermal mechanism and a mouthpiece through which the aerosolized solution is inhaled.

### PRIORITY CLAIM

This non-provisional patent application claims the benefit of U.S. Provisional Application No. 63/808,042 filed on May 19, 2025, the entirety of which is incorporated herein by reference.

### FIELD

The present disclosure relates generally to devices and methods of inhalation. More particularly, the present disclosure relates to portable inhalation devices. Such devices may comprise a non-heating aerosol device designed to deliver hydration and performance-enhancing compounds such as electrolytes, caffeine, adaptogens, vitamins, and nootropics for use in athletic, social, and cognitive enhancement settings.

### BACKGROUND

Certain traditional methods of delivering hydration and stimulants involve oral consumption of liquids or supplements. With such methods, administration can often be inconvenient during physical activities or social events. There is, therefore, a need for a portable, efficient, and rapid delivery system for electrolytes, caffeine, and other energy-enhancing compounds that can be used discreetly and conveniently in various settings.

### SUMMARY

According to an exemplary arrangement, ₐn inhalation device comprising a housing, a reservoir configured within the housing, an aqueous solution contained within the reservoir, a non-thermal mechanism that aerosolizes the aqueous solution, a power source operably coupled to the non-thermal mechanism and a mouthpiece through which the aerosolized solution is inhaled.

In one arrangement, the non-thermal mechanism comprises an atomizer.

In one arrangement, the non-thermal mechanism comprises a nebulizer.

In one arrangement, the nebulizer comprises a mesh nebulizer.

In one arrangement, the non-thermal mechanism comprises an ultrasonic mechanism.

In one arrangement, the aqueous solution comprises elements selected from the group of electrolytes, caffeine, and flavoring agents.

In one arrangement, the aqueous solution further comprises an optional additive.

In one arrangement, the aqueous solution comprises about 30 mg to about 100 per reservoir.

In one arrangement, the solution comprises an isotonic solution.

In one arrangement, the device further comprising control circuitry that regulates the non-thermal mechanism that aerosolizes the aqueous solution.

In one arrangement, the control circuitry is operably coupled to a charging circuit and a power source.

In one arrangement, the power source is operably coupled to a charging port.

In one arrangement, the charging port comprises a USB-C Charging Port.

In one arrangement, the optional additives are selected from a group including vitamins, adaptogens, and amino acids.

In one arrangement, the power source comprises a rechargeable power source.

In one arrangement, the device of further comprising formulation cartridge.

In one arrangement, the formulation cartridge comprises a flavor cartridge.

In one arrangement, the device further comprising a vapor pathway configured between the non-thermal mechanism that aerosolizes the aqueous solution and a mouthpiece of the inhalation device.

In one arrangement, the reservoir comprises a refillable reservoir.

In one arrangement, the device further comprising a refill port configured to allow the reservoir to be refilled.

In one arrangement, the inhalation device comprises an inhale-activated device.

In one arrangement, the inhalation device comprises a manually activated inhalation device.

In one arrangement, the inhalation device further comprises a plurality of activation indicators.

In one arrangement, the activation indicators comprise a plurality of light emitting devices (LEDs).

In one arrangement, the inhalation device comprises an inhale-activated mechanism. In such an arrangement, the inhale-activated mechanism may comprise an airflow or pressure sensor operably coupled to a control circuitry. In one arrangement, such a configuration may be located in a mouthpiece.

The features, functions, and advantages can be achieved independently in various embodiments of the present disclosure or may be combined in yet other embodiments in which further details can be seen with reference to the following description and drawings.

### BRIEF DESCRIPTION OF THE DRAWINGS

The novel features believed characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 illustrates exemplary illustration of an inhalation device, according to one arrangement;

FIG. 2 illustrates a cross-sectional view of an inhalation device, according to one arrangement;

FIG. 3 illustrates an exemplary illustration of a circuit diagram for use with an inhalation device, according to one arrangement;

FIG. 4 illustrates an exemplary flow diagram for use with an inhalation device, according to one arrangement; and

FIG. 5 illustrates another exemplary illustration of a circuit diagram for use with an inhalation device, according to one arrangement.

### DETAILED DESCRIPTION

The following detailed description describes various features and functions of the disclosed systems and methods with reference to the accompanying figures. The illustrative system and method embodiments described herein are not meant to be limiting. It may be readily understood that certain aspects of the disclosed systems and methods can be arranged and combined in a wide variety of different configurations, all of which are contemplated herein.

Further, unless context suggests otherwise, the features illustrated in each of the figures may be used in combination with one another. Therefore, the figures should be generally viewed as component aspects of one or more overall implementations, with the understanding that not all illustrated features are necessary for each implementation.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

The present disclosure provides a portable inhalation device that aerosolizes a solution containing electrolytes, caffeine, and other performance-enhancing compounds. In one preferred arrangement, the inhalation device is designed for use during physical activities and social events to deliver hydration and energy-enhancing compounds through inhalation, offering a convenient alternative to traditional consumption methods.

For example, the present disclosure provides a compact, vape-style inhalation device utilizing a non-combustive aerosol generation method such as mesh or ultrasonic nebulization. It delivers an aqueous-based solution containing a combination of electrolytes, caffeine, vitamins, amino acids, adaptogens, and/or nootropics. The device is optimized for use in fitness settings (pre/intra/post-workout), social environments (e.g. nightclubs, festivals), and cognitively demanding contexts (e.g. studying, traveling, shift work).

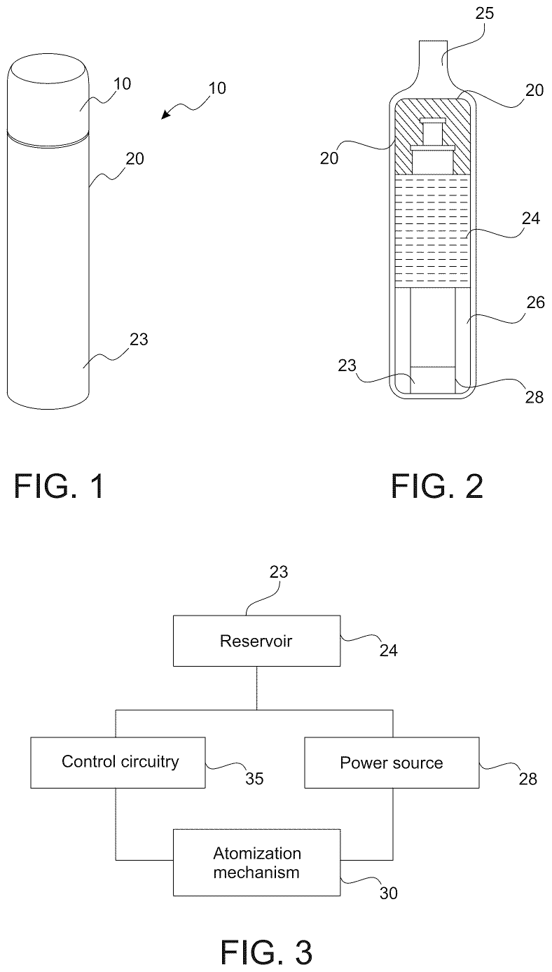

FIG. 1 illustrates an exemplary illustration of an inhalation device 10, according to one arrangement. Such an inhalation device 10 may comprises a removable cap 40, a housing 20, a reservoir 28 configured within the housing 20, an aqueous solution 24 contained within the reservoir, a non-thermal mechanism that aerosolizes the aqueous solution, and a power source operably coupled to the non-thermal mechanism and a mouthpiece 25 through which the aerosolized solution is inhaled.

### Housing

As illustrated in FIG. 1, the inhalation device 10 comprises a housing 20. FIG. 2 illustrates a cross-sectional view of one embodiment of an inhalation device, such as the device 10 illustrated in FIG. 1. In one arrangement, this housing 20 comprises a compact, cylindrical body resembling conventional disposable vape pens, facilitating portability and user familiarity. However, as those of ordinary skill in the art will recognize, alternative housing arrangements and housing configurations may be utilized as well.

In one preferred arrangement, the inhalation device's housing 20 comprises a sleek and cylindrical, mimicking the slim profile of a vaping pen for discreet portability. In this illustrative arrangement, the housing's slender design, measuring about 15-20 mm in diameter, fits comfortably in the hand or pocket. Different types of housing materials may be utilized. As just one example, the housing 20 may be crafted from lightweight medical-grade plastic or anodized aluminum. With such an arrangement, the housing 20 maintains a minimalist aesthetic without sacrificing durability. The streamlined form factor, approximately 120 mm in length, ensures ease of use and storage. With smooth, curved edges and a compact footprint, the device blends seamlessly into daily life.

For examples, such a housing 20 can be made from a variety of materials, each offering certain benefits. Aluminum is a popular choice due to its lightweight nature and durability. It provides a sleek, premium feel and excellent heat dissipation. Polycarbonate is another common material, known for its impact resistance and flexibility, making it ideal for rugged smartphones. Glass is often used for its aesthetic appeal and smooth finish, allowing for wireless charging capabilities, though it can be more fragile. Ceramic offers a luxurious feel and is highly scratch-resistant, though it can be more expensive and heavier. Lastly, carbon fiber is used for its strength and lightweight properties, providing a high-tech look and feel while being extremely durable. Each material contributes to the overall functionality and design of the device housing 20.

### Mouthpiece

As illustrated in FIG. 2, in one preferred arrangement, the inhalation device 10 may further comprise a mouthpiece 25 designed for comfortable inhalation of the aerosolized solution. For example, in one arrangement, the inhalation device 10 comprises a removable mouthpiece. Such a removable mouthpiece might be advantageous for multiple reasons. For example, a removable mouthpiece can enhance hygiene, allowing users to easily detach and clean it with warm water and mild soap to prevent bacterial buildup. It enables personalization, as users can replace the mouthpiece with one tailored for comfort, such as a different size or shape for pediatric or adult users. The detachable design facilitates maintenance, making it simple to inspect and clear any residue from the medication chamber or mesh.

In addition, a removable mouthpiece also supports versatility, as the mouthpiece can be swapped for a mask adapter for patients who prefer or require mask-based inhalation. In addition, a removable mouthpiece can extend the device's lifespan, as worn or damaged mouthpieces can be replaced without discarding the entire unit. Moreover, a removable mouthpiece also promotes portability, as the mouthpiece can be detached and stored separately, reducing the device's overall size for travel.

In an alternative arrangement, the inhalation device 10 comprises a mouthpiece that may comprise a non-removable mouthpiece. A non-removable mouthpiece in the inhalation device 10 can offer several benefits that enhance reliability, safety, and user experience. Its integrated design can help to ensure a secure, airtight connection to the nebulizer, minimizing the risk of leaks or misalignment that could compromise aerosol delivery. This fixed configuration can enhance durability by eliminating detachable parts that could wear out, break, or be lost, making it ideal for frequent or long-term use.

A non-removable mouthpiece can also simplify operation, as inhalation device users, including those with limited dexterity like elderly or pediatric patients, do not need to attach or detach components, reducing the chance of user error. It also maintains the device's sleek, vape-like aesthetic, contributing to a compact and discreet form factor suitable for portable use. Additionally, the permanent attachment can be designed to optimize airflow and aerosol deposition, ensuring consistent and efficient delivery of medication to the lungs.

### Reservoir

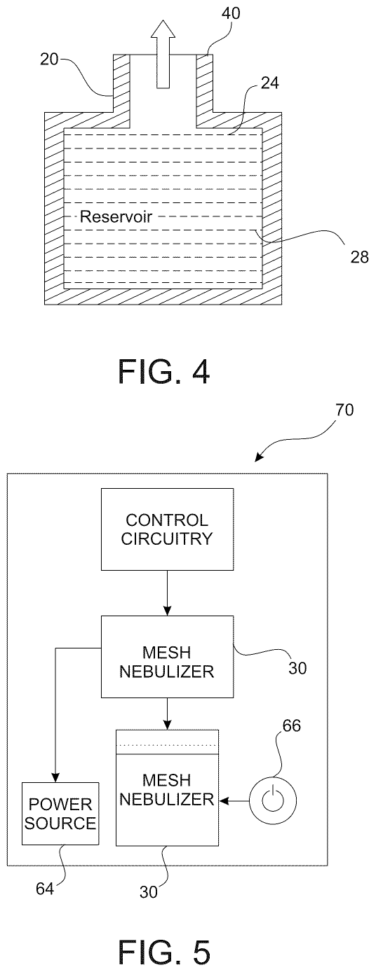

As illustrated in FIGS. 2-4, the inhalation device 10 may also comprise a reservoir 28. And like the mouthpiece 25, the reservoir 28 may comprise a removable and/or a non-removable or a refillable reservoir. In one arrangement, the reservoir 28 may be located near a proximal end of the inhalation device 10. However, as those of ordinary skill in the relevant art will recognize, alternative reservoir locations may be utilized as well.

A removable reservoir in the inhalation device 10 offers numerous advantages that enhance user convenience and device longevity. It allows for easy refilling, enabling users to swiftly switch between different medications or alternative solutions as prescribed without complex procedures. Cleaning is simplified, as the reservoir 24 can be detached and thoroughly washed with warm water and mild soap, preventing bacterial contamination or residue buildup that could impair performance. Users can replace a damaged or worn reservoir without discarding the entire inhalation device 10, making it a cost-effective solution that extends the device's lifespan. The detachable design also supports customization, as reservoirs of varying capacities, such as 1 mL or 2 mL, can be used to suit specific treatment needs.

Additionally, a removable reservoir enhances portability, as the reservoir can be removed and stored separately to prevent leaks during travel, and it facilitates inspection for clogs or sediment that might affect the mesh nebulizer's efficiency.

In alternative arrangements, the inhalation device 10 may comprises a non-removable reservoir. For example, a non-removable reservoir can provide certain benefits that prioritize reliability and ease of use. Its sealed, integrated design can minimize the risk of leaks or improper reattachment, ensuring consistent solution delivery. The absence of detachable parts can also enhance durability, reducing the likelihood of components wearing out, breaking, or being lost during regular use or travel. This fixed reservoir can simplify operation, making the inhalation device 10 more accessible for individuals, such as elderly or pediatric patients, who may find assembling components challenging. It also reduces user error by eliminating the need to align or secure a detachable reservoir, guaranteeing reliable performance. Furthermore, a non-removable reservoir can support a more robust connection to the mesh nebulizer, optimizing aerosol delivery and minimizing maintenance needs, while contributing to a sleeker, more compact form factor that aligns with the vape-like aesthetic for discreet portability.

### Refill Port

As illustrated in FIGS. 1-3, in one arrangement, the inhalation device 10 further comprising a refill port 23 that is configured to allow the reservoir 28 of the inhalation device 10 to be refilled. Incorporating a refill port 23 into the inhalation device 10 offers a number of potential advantages by streamlining the process of loading medication or solution into the reservoir 28. The refill port 23 allows users to easily add liquid solution 24, such as isotonic saline or desired solutions, without disassembling the device 10, enhancing convenience and reducing the risk of spills. Its design, typically featuring a secure, leak-proof cap or valve, ensures safe and precise refilling, which is particularly beneficial for users with limited dexterity, such as elderly or pediatric patients.

The refill port 23 supports versatility, enabling the use of various medications or solutions compatible with the vibrating mesh nebulizer, accommodating different treatment needs. By simplifying the refilling process, the port encourages consistent use and adherence to therapy schedules, improving treatment outcomes for conditions like asthma or COPD. Additionally, a well-designed refill port maintains the device's sleek, vape-like aesthetic while ensuring the reservoir remains sealed during operation, preventing contamination and preserving the medication's integrity.

### Solution

In one arrangement, the reservoir 28 is filled with an amount of an aqueous solution 24 and this aqueous solution, in one arrangement, comprises elements selected from the group of electrolytes, caffeine, and flavoring agents. In one arrangement, the aqueous solution 24 further comprises an optional additive. In yet another arrangement, the optional additives are selected from a group including vitamins, adaptogens, and amino acids.

In one preferred arrangement, the device 10 consists of a pen-sized cylindrical housing made of plastic or aluminum alloy. In one preferred arrangement, within the housing is a reservoir 28 holding 5-8 mL of an aqueous formulation 24. The formulation 24 may contain any combination of the following:
Electrolytes (e.g., sodium chloride, potassium citrate, magnesium sulfate)
Caffeine (30-100 mg per reservoir)
Adaptogens (e.g., Rhodiola Rosea, Ginseng)
Amino Acids (e.g., BCAAs such as leucine, isoleucine, valine)
Nootropics (e.g., L-theanine, citicoline)
Vitamins (e.g., B12, C)
Flavoring agents (natural mint, citrus, etc.)

Various amounts or quantities of the solution may be provided. As just one example, in one arrangement, the aqueous solution 24 comprises about 30 mg to about 100 per reservoir. However, as those of ordinary skill in the relevant art will recognize, alternative solution amounts may be utilized as well.

In one arrangement, the solution 24 comprises an isotonic solution. For example, using an isotonic solution in the inhalation device's reservoir 28 offers several potential advantages for effective and comfortable respiratory therapy. An isotonic solution, with a salinity matching that of human bodily fluids (approximately 0.9% sodium chloride), minimizes irritation to the respiratory mucosa, ensuring patient comfort during inhalation. This compatibility reduces the risk of bronchoconstriction or discomfort, which is particularly beneficial for patients with sensitive airways, such as those with asthma or COPD. Isotonic solutions maintain cellular integrity in the lungs, preventing osmotic stress that could occur with hypotonic or hypertonic solutions, thus promoting safer drug delivery. They also enhance the stability of aerosolized medications, ensuring consistent particle size and effective deposition in the lower airways. Furthermore, isotonic solutions are widely compatible with a variety of medications, making them versatile for use in mesh nebulizers without compromising therapeutic efficacy.

### Non-Thermal Mechanism

As illustrated in FIGS. 3 and 5, the inhalation device 10 comprises a non-thermal element 30, such as an atomization mechanism or a mesh nebulizer. Utilizing a non-thermal element 30, such as a vibrating mesh, to create a mist of the solution in the inhalation device 10 offers certain advantages for both efficacy and safety. Unlike thermal methods, a non-thermal vibrating mesh nebulizer generates aerosol through high-frequency mechanical vibrations, preserving the chemical stability of sensitive medications like proteins or steroids that could degrade under heat.

This approach helps to ensure consistent production of fine droplets (1-5 μm), ideal for deep lung penetration, without altering the solution's therapeutic properties. Non-thermal mist creation is energy-efficient, allowing for prolonged battery life in a portable, vape-like inhalation device, enhancing user convenience. Additionally, non-thermal devices can operate quietly (typically 25-30 dB), making the device 10 discreet for public use compared to louder jet nebulizers. Lastly, the absence of heat eliminates the risk of thermal injury to the user or degradation of the device's components, improving safety and durability.

In one arrangement, the non-thermal mechanism 30 comprises an atomizer. _{I}n the inhalation device 10, the atomizer, specifically a vibrating mesh nebulizer, operates by transforming the liquid medication into a fine mist for effective respiratory delivery. In one arrangement, a piezoelectric actuator, typically a ceramic ring, vibrates at a high frequency (e.g., 98.5 kHz) when an electric current is applied, creating ultrasonic waves. These vibrations are transmitted to a thin, micro-porous mesh membrane, often made of palladium-nickel, with thousands of tiny apertures (3-5 μm in diameter). As the liquid solution 24 within the reservoir 28 contacts the vibrating mesh, the solution 24 is forced through these apertures, breaking into consistent, respirable droplets with a mass median aerodynamic diameter (MMAD) of approximately 3-5 μm. This fine mist is then inhaled through the mouthpiece, ensuring deep lung penetration without the use of heat or propellants. The atomizer's precise control over droplet size and quiet operation make it ideal for a discreet, vape-like inhalation device.

In one arrangement, the non-thermal mechanism 30 comprises a nebulizer. For example, in one arrangement, the nebulizer comprises a mesh nebulizer. A nebulizer in this inhalation device 10, specifically a vibrating mesh nebulizer, functions by converting liquid medication into a fine aerosol mist for respiratory therapy. In one arrangement, a piezoelectric actuator vibrates at a high frequency (e.g., 98.5 kHz) when activated by an electric current, generating ultrasonic waves. These waves drive a micro-porous mesh membrane, typically made of palladium-nickel, featuring thousands of tiny apertures (3-5 μmin diameter). The solution 24 in the reservoir 28 is pushed through these apertures as the mesh vibrates, forming droplets with a mass median aerodynamic diameter (MMAD) of 3-5 μm, ideal for deep lung delivery. The mist is then inhaled through the mouthpiece 25, delivering the solution 24 efficiently to the airways without heat or propellants. This quiet, non-thermal process ensures solution stability and discreet operation, aligning with the device's sleek, vape-like design.

As the term is used herein, ₜhe nebulizer is the broader system within the inhalation device that converts liquid solution into a fine aerosol mist for respiratory delivery, encompassing components like the medication reservoir, mesh, and delivery mechanism. The atomizer, specifically, is the core component of the nebulizer—typically the vibrating mesh and piezoelectric actuator—that physically transforms the liquid into mist by forcing it through micro-porous apertures. In this vape-like device, the atomizer refers to the mesh nebulizer's aerosol-generating mechanism, which produces droplets with a mass median aerodynamic diameter (MMAD) of 3-5 μm. While the terms are sometimes used interchangeably in casual contexts, the nebulizer includes the atomizer plus additional elements like the mouthpiece and housing. Therefore, the atomizer is a critical part of the nebulizer but does not encompass the entire system. This distinction ensures clarity in understanding the device's operation and design.

### Control Circuitry

In one arrangement, and as illustrated in FIGS. 3, 5 and 6, the inhalation device 10 further comprises control circuitry 35 that regulates the non-thermal mechanism that aerosolizes the aqueous solution. As just one example, the control circuitry 35 may comprise a microprocessor or processor based control circuitry.

In one preferred arrangement, the processor comprises the central unit that executes instructions and processes data. In one preferred arrangement, the processor interprets user inputs, runs applications, and manages data flow between components, ensuring smooth operation.

For example, in one arrangement, the processor may comprise a processor such as that used in certain hand held electronic devices and generally referred to as the Qualcomm Snapdragon series. This is a processor generally known for its high performance and efficiency. The processor, or SoC (System on Chip), integrates the CPU, GPU, and other essential components. It works closely with the memory (RAM and storage) to quickly access and process data, ensuring smooth multitasking and application performance. The processor also interfaces with human interface devices or activation devices (i.e., LEDS) as herein described like the touchscreen, interpreting touch inputs and gestures to provide a responsive user experience.

In one arrangement, the inhalation device comprises an inhale-activated mechanism. In such an arrangement, the inhale-activated mechanism may comprise an airflow or pressure sensor operably coupled to a control circuitry. In one arrangement, such a configuration may be located in a mouthpiece. However, as those of ordinary skill in the art will recognize, alternative locations may be utilized as well.

### Power Source

In one arrangement, the control circuitry is operably coupled to a charging circuit and a power source. In one arrangement, the power source is operably coupled to a charging port. Incorporating a charging port into the inhalation device 10 can enhances its portability and user convenience by enabling a rechargeable power source. A USB-C charging port, for instance, allows the device's lithium-ion battery (e.g., 3.7V, 500-1000 mAh) to be recharged quickly and easily using widely available cables and power sources, such as wall adapters or power banks. This can eliminate the need for frequent battery replacements, reducing long-term costs and environmental waste, which is particularly beneficial for daily users with chronic respiratory conditions. The charging port can support the device's compact, vape-like design by maintaining a sleek profile without bulky external battery compartments. It also can help to ensure consistent performance, as a rechargeable battery can power multiple treatments (e.g., 5-10 sessions of 5-10 minutes) on a single charge, ideal for on-the-go use. Additionally, a charging port with LED indicators can provide real-time feedback on battery status, helping users plan recharges and avoid interruptions during therapy.

In one arrangement, the charging port comprises a USB-C Charging Port. A USB-C charging port in the inhalation device offers certain advantages, enhancing both functionality and user experience. Its universal compatibility allows the device to be recharged using widely available USB-C cables and power sources, such as laptops, wall adapters, or power banks, ensuring convenience for users at home or on the go. The USB-C port supports fast charging, efficiently powering the device's lithium-ion battery (e.g., 3.7V, 500-1000 mAh) to deliver multiple treatment sessions (5-10 minutes each) after a short charging period. This design helps to eliminate the need for disposable batteries, reducing long-term costs and environmental impact, which is ideal for patients with chronic respiratory conditions requiring frequent use. The compact and reversible nature of USB-C maintains the sleek, vape-like aesthetic of the inhalation device while ensuring easy and secure connections. Additionally, integrating a USB-C port with LED indicators provides clear battery status updates, helping users manage recharging schedules to avoid disruptions during therapy.

### Power Source

The power source 28, typically a rechargeable battery, supplies electrical power to the inhalation device 10. The power source 28 may be connected to the processor and other components, ensuring they receive the necessary power to function. The power source 28 enables the device 10 to operate independently of external power supplies.

The mobile devices 10 as disclosed herein can use various types of power sources to ensure they operate efficiently and meet user needs. Provided below is a general overview of the different types of power sources that may be used in various arrangements.

### Lithium-Ion (Li-Ion) Batteries

The most common type of battery used in modern electric devices. Li-Ion batteries are known for their high energy density, lightweight, and long lifespan. They provide a high energy density allows for longer battery life. They are also lightweight and compact, making them ideal for slim smartphone designs. They also have a low self-discharge rate, meaning they retain charge well when not in use.

### Lithium-Polymer (Li-Po) Batteries

These are similar to Li-Ion batteries but use a polymer electrolyte instead of a liquid one. They can be molded into various shapes and sizes. They comprise a Flexible form factor allows for innovative smartphone designs. Generally safer and less prone to leaking compared to Li-Ion batteries. Lightweight with a high energy density.

### Replaceable Batteries

These batteries that can be easily removed and replaced by the user. This design was more common in older smartphones. Users can carry spare batteries and replace them when needed, extending the device's usage time. Easier to replace a degraded battery without needing professional assistance.

### Nickel-Metal Hydride (NiMH) Batteries

An older type of rechargeable battery that was used in early mobile phones. More environmentally friendly compared to older nickel-cadmium (NiCd) batteries. Can be recharged many times without significant memory effect.

### User Activated

In one arrangement, the inhalation device comprises an inhale-activated device. In an alternative arrangement, the inhalation device comprises a manually activated inhalation device. In one arrangement, the inhalation device 10 may incorporate a user activation device, such as a breath-actuated mechanism, to enhance efficiency and ease of use compared to a manually activated device, ensuring optimal delivery of medication. Unlike manual activation, which may require the user to press a button or switch to initiate aerosol production, a breath-actuated system employs a sensor, typically a pressure or airflow sensor, to detect the user's inhalation through the mouthpiece. In one arrangement, the inhale-activated mechanism comprises an airflow or pressure sensor that is operably coupled to the control circuitry and located in the mouthpiece. When the sensor registers a sufficient inspiratory flow (e.g., 15-30 L/min), it triggers the piezoelectric actuator to vibrate the mesh, generating a fine mist (3-5 μm MMAD) precisely synchronized with the user's breathing.

This synchronization can minimize solution waste, as aerosol is produced only during inhalation, maximizing the respirable fraction delivered to the lungs. Breath actuation is particularly advantageous for patients with limited coordination or dexterity, such as children or the elderly, who may struggle with the timing required for manual activation. The system can be powered by the device's rechargeable lithium-ion battery, ensuring seamless integration into the compact, vape-like design without adding bulk.

Additionally, the user activation device can include a microcontroller as herein described to adjust the sensitivity of the sensor, accommodating different breathing patterns (e.g., adult vs. pediatric tidal volumes). This feature enhances versatility, making the inhalation device 10 suitable for a wide range of users with conditions like asthma or COPD. Unlike manual devices, which may lead to inconsistent dosing if not timed correctly, breath actuation ensures consistent and efficient medication delivery, improving therapeutic outcomes. Furthermore, the absence of manual controls simplifies the user interface, reducing the learning curve and aligning with the device's sleek, discreet aesthetic while maintaining reliable performance.

### Manually Activated

In an alternative arrangement, ₜhe inhalation device 10 comprises a manually activated device, such as a push-button or switch, to initiate aerosol production, offering a straightforward and user-controlled approach to respiratory therapy. With this design, the user manually presses a button to activate the piezoelectric actuator, which vibrates the micro-porous mesh membrane (e.g., palladium-nickel with 3-5 μm apertures) at an ultrasonic frequency (e.g., 98.5 kHz), transforming the liquid solution 24 in the reservoir 28 into a fine mist with a mass median aerodynamic diameter (MMAD) of 3-5 μm. This manual activation can allow users to consciously control when the aerosol is produced, which can be beneficial for those who prefer deliberate timing or need to coordinate inhalation with specific breathing patterns.

In one arrangement, such a manually activated system may be powered by the device's rechargeable lithium-ion battery (e.g., 3.7V, 500-1000 mAh), ensuring consistent performance across multiple treatment sessions (5-10 minutes each) without adding complexity to the compact, vape-like form factor. A single-button interface can simplify device operation, making it accessible for users of varying ages and technical abilities, including those with conditions like asthma or COPD. The manually activated device can include an LED indicator to confirm activation and provide feedback on battery status, enhancing user confidence during operation.

Unlike breath-actuated systems, which require sensors and may not suit patients with weak inspiratory flow, manual activation ensures functionality regardless of breathing strength, offering reliability for a broader range of users. The design also reduces manufacturing costs compared to sensor-based systems, as it eliminates the need for additional components like pressure or airflow sensors and their associated microcontrollers. However, users must coordinate their inhalation with button activation to optimize aerosol delivery, which may require brief training to achieve consistent dosing. Overall, the manually activated mechanism aligns with the device's sleek, portable design while providing a cost-effective, reliable, and user-friendly option for delivering medication efficiently to the lungs.

### Memory

As illustrated, the control circuity 35 may comprise a memory that stores data and executable instructions. It is operably coupled to the processor as herein described, providing the memory with certain necessary information to execute certain tasks. In one preferred arrangement, the memory holds or stores the operating system, applications, and user data, enabling quick access and efficient processing.

The exemplary inhalation device 10 may use various types of memory to store data and execute tasks. These different types of memory that may be used in the exemplary device 10 are summarized below.

### Non-Removable Memory (Internal Storage)

Flash Memory (NAND Flash): This is the primary storage used in hand held electronic devices. It is non-volatile, meaning it retains data even when the device is powered off. NAND flash memory is fast and reliable, making it ideal for storing the operating system, applications, and user data.

EEPROM (Electrically Erasable Programmable Read-Only Memory): EEPROM is used for storing small amounts of data that must be saved when power is removed, such as firmware and configuration settings. It is slower than NAND flash but offers the advantage of being reprogrammable.

### Removable Memory

MicroSD Cards: These are small, removable memory cards that can be inserted into the device 10 to expand its storage capacity. They are available in various capacities, ranging from a few gigabytes (GB) to several terabytes (TB).

### Volatile Memory (RAM)

DRAM (Dynamic Random-Access Memory): This type of memory is used for temporary data storage while the device 10 is in use. It is volatile, meaning it loses data when power is turned off. DRAM is crucial for multitasking and running applications smoothly.

### Non-Volatile Memory

ROM (Read-Only Memory): ROM is used to store the smartphone's firmware, which includes the bootloader and system software. It is non-volatile and cannot be easily modified by the user. ROM ensures that the device 10 can boot up and operate correctly.

NOR Flash: This type of flash memory is used for storing firmware and boot code. It offers fast read speeds and is reliable for storing critical system data.

### Storage Capacity Types

eMMC (Embedded MultiMediaCard): eMMC is a type of flash storage used in budget and mid-range smartphones. It integrates the memory controller and NAND flash memory into a single package. eMMC is slower than UFS but still provides adequate performance for everyday tasks.

### Input Interface

Referring back to FIGS. 1-4, the inhalation device 10 may further comprise an input interface. In one preferred arrangement, the input interface comprises one or more components such as touchscreens, buttons, and microphones, which receive user inputs. As illustrated, the input interface is operatively coupled to the control circuitry and/or processor, which interprets these inputs and executes corresponding actions. In one preferred arrangement, the input interface allows users to interact with the inhalation device 10, navigate menus, and control functions.

The presently disclosed devices 10 use a variety of human interface devices (HIDs) or activation features to enable user interaction. Below is an overview of the different types of HIDs that may be used in device 10 illustrated in FIGS. 1-4.

Capacitive Touchscreen: The most common type, it detects touch through the electrical properties of the human body. It supports multi-touch gestures, allowing for pinch-to-zoom, swiping, and other gestures.
Resistive Touchscreen: Less common in modern smartphones, it detects touch through pressure applied to the screen. It can be used with a stylus or finger but does not support multi-touch.
Power Button: Used to turn the device on/off and wake it from sleep mode.
Volume Buttons: Adjust the audio levels for calls, media, and notifications.
Home Button: Found on some smartphones, it provides quick access to the home screen and may include a fingerprint sensor for biometric authentication.
Side Buttons: Additional buttons that can be customized for various functions, such as launching the camera or activating voice assistants.
Mute Switch: Found on some smartphones, it allows users to quickly mute or unmute the device.
Rotary Knobs: Rare in smartphones but can be found in some specialized devices for adjusting settings like volume or brightness.

### Graphical User Interface (GUI)

Icons and Widgets: Visual elements on the home screen and app drawer that provide quick access to applications and information.
Virtual Keyboards: On-screen keyboards that appear when text input is needed. They can be customized with different layouts and languages.
Navigation Gestures: Swipe and tap gestures used to navigate the operating system, switch between apps, and access settings.
Notification Shade: A pull-down menu that displays notifications, quick settings, and shortcuts.

### Biometric Sensors

Fingerprint Scanner: Integrated into the home button, power button, or under the display, it allows for secure authentication and unlocking of the device.
Face Recognition: Uses the front-facing camera and advanced algorithms to recognize the user's face for unlocking the device and authorizing transactions.
Iris Scanner: Uses infrared light to scan the unique patterns in the user's iris for secure authentication.

### Voice Input

Microphone: Captures voice commands for virtual assistants like Siri, Google Assistant, and Alexa. It also enables voice typing and voice search.
Voice Assistants: Software that interprets voice commands to perform tasks, answer questions, and control smart home devices.

### Haptic Feedback

Vibration Motor: Provides tactile feedback to simulate the sensation of pressing physical buttons or to alert the user through vibrations.

The description of the different advantageous embodiments has been presented for purposes of illustration and description and is not intended to be exhaustive or limited to the embodiments in the form disclosed. Modifications and variations will be apparent to those of ordinary skill in the art. Further, different advantageous embodiments may provide different advantages as compared to other advantageous embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

I claim:

1. An inhalation device comprising:
a housing
a reservoir configured within the housing;
an aqueous solution contained within the reservoir;
a non-thermal mechanism that aerosolizes the aqueous solution;
a power source operably coupled to the non-thermal mechanism; and
a mouthpiece through which the aerosolized solution is inhaled.

2. The device of claim 1, wherein the non-thermal mechanism comprises an atomizer.

3. The device of claim 1, wherein the non-thermal mechanism comprises a nebulizer.

4. The device of claim 3, wherein the nebulizer comprises a mesh nebulizer.

5. The device of claim 1, wherein the non-thermal mechanism comprises an ultrasonic mechanism.

6. The device of claim 1, wherein the aqueous solution comprises elements selected from the group of electrolytes, caffeine, and flavoring agents

7. The device of claim 6, wherein the aqueous solution further comprises an optional additive.

8. The device of claim 6, wherein the aqueous solution comprises about 30 mg to about 100 per reservoir.

9. The device of claim 1, wherein the solution comprises an isotonic solution.

10. The device of claim 1, further comprising control circuitry that regulates the non-thermal mechanism that aerosolizes the aqueous solution.

11. The device of claim 10, wherein the control circuitry is operably coupled to a charging circuit and a power source.

12. The device of claim 11, wherein the power source is operably coupled to a charging port.

13. The device of claim 10, wherein the charging port comprises a USB-C Charging Port.

14. The device of claim 1, wherein the optional additives are selected from a group including vitamins, adaptogens, and amino acids.

15. The device of claim 11, wherein the power source comprises a rechargeable power source.

16. The device of claim 1, further comprising formulation cartridge.

17. The device of claim 16, wherein the formulation cartridge comprises a flavor cartridge.

18. The device of claim 1, further comprising a vapor pathway configured between the non-thermal and a mouthpiece of the inhalation device.

19. The device of claim 1, wherein the reservoir comprises a refillable reservoir.

20. The device of claim 1, further comprising a refill port configured to allow the reservoir to be refilled.

21. The device of claim 1, wherein the inhalation device comprises an inhale-activated device.

22. The device of claim 1, wherein the inhalation device comprises a manually activated inhalation device.

23. The device of claim 1, wherein the inhalation device further coprises a plurality of activation indicators.

24. The device of claim 23, wherein the activation indicators comprise a plurality of light emitting devices (LEDs).

25. The device of claim 1, wherein the device comprises an inhale-activated mechanism comprising an airflow or pressure sensor operably coupled to a control circuitry and is located in a mouthpiece.
